(19) United States
(12) Patent Application Publication
(10) Pub. No.: US 2011/0290270 A1
(43) Pub. Date: Dec. 1, 2011

(54) Composition and Method for Aesthetic Repair of Fingernails and Toenails

(76) Inventor: Gerry M. Schwartz, Miami, FL (US)

(21) Appl. No.: 13/115,489

(22) Filed: May 25, 2011

Related U.S. Application Data
(60) Provisional application No. 61/348,011, filed on May 25, 2010.

(51) Int. Cl. A61Q 3/02; A61K 8/02; A61K 8/18

(52) U.S. Cl. 132/200; 424/61; 424/401

(57) ABSTRACT

A composition and method is provided to repair fingernail and toenail anomalies. The composition is a carrier and reflective material that, when applied to a fingernail or toenail anomaly, fills said anomaly and reflects the nearby color, thereby reducing or eliminating the visual presence of the anomaly.

### CLAIM FOR PRIORITY

This application claims the benefit and priority of U.S. Provisional Patent Application Ser. No. 61/348,011 filed May 25, 2010 which is incorporated herein in its entirety.

### BACKGROUND OF THE INVENTION

Human fingernails are made up of layers of keratin—a protein that's also found in both hair and skin. Each nail is comprised of several parts.

The structure known as the fingernail is divided into six specific parts—the root, nail bed, nail plate, eponychium (cuticle), perionychium, and hyponychium.

Each of these structures has a specific function, and if disrupted can result in an abnormal appearing fingernail.

The root of the fingernail is also known as the germinal matrix. This portion of the nail is actually beneath the skin behind the fingernail and extends several millimeters into the finger itself. The fingernail root produces most of the volume of the nail and the nail bed. This portion of the nail does not have any melanocytes, or melanin producing

The nail plate is the actual fingernail, made of translucent keratin. The pink appearance of the nail comes from the blood vessels underneath the nail. The underneath surface of the nail plate has grooves along the length of the nail that help anchor it to the nail bed.

Nail folds are the skin that frames each of the fingernails on three sides.

A nail bed is the skin beneath the nail plate.

The cuticle of the fingernail is also called the eponychium. The cuticle is situated between the skin of the finger and the nail plate fusing these structures together and providing a waterproof barrier. Cuticle tissue overlaps the nail plate at the base of the nail.

The lunula is the whitish, half-moon shape at the base of your nail.

The perioncyhium is the skin that overlies the nail plate on its sides. It is also known as the paronychial edge. The perionychium is the site of hangnails, ingrown nails, and an infection of the skin called paronychia.

The hyponychium is the area between the nail plate and the fingertip. It is the junction between the free edge of the nail and the skin of the fingertip, also providing a waterproof barrier.

Cracks, chips, or other surface anomalies are often viewed unfavorably.

Fingernail polish is a lacquer or other coating applied to toenails and fingernails for appearance, but also as nail protection.

The present invention is a composition for repairing the various anatomical parts of a fingernail and repairing cracks or chips in fingernail polish.

Nail polish consists of a pigment and a flowable resin material. The nail polish is painted on a surface of a nail using a brush and when allowed to dry, the pigment becomes embedded in the resin.

Incoming light, typically light of a broad wavelength spectrum such as daylight, contacts the surface of the dried polish, and is partially transmitted into the polish and partially reflected. The wavelengths of light that are transmitted into the polish are selectively absorbed or reflected by the pigment, to a large degree, and the resin to a smaller degree. The wavelengths not absorbed by the pigment material are reflected and perceived as that color by the eye. The nail polish has an index of refraction, based on the components, including at least the pigment and resin material of the nail polish.

When a nail, having nail polish previously applied, becomes “chipped”, a section of the nail polish on a nail is partially or completely dislodged from the surface of the nail resulting in a “chip”. The chipped section likely will be seen by an observer, as a defect in the polished nail.

Preferably, the chipped surface is repainted with nail polish of like pigment color to match the nail polish remaining on the surface. This becomes inconvenient when the applied color of polish is unavailable, or not readily available e.g. is at home and not in the user's possession.

### BRIEF SUMMARY OF THE INVENTION

The present invention is a composition and method to repair fingernail and toenail anomalies. An anomaly, as used herein, refers to cracks, crevices, holes, and the like in any part of the nail surface. Additionally, an anomaly, as used herein can refer to any crack, crevice, or hole in a coating applied to the nail. Coatings, as known in the art, encompass many materials and include, but limited to polishes, acrylics, combinations thereof, and the like. The composition is a carrier and reflective material that, when applied to a fingernail or toenail anomaly, fills said anomaly and reflects a color that is the same color of the nearby coating color, thereby reducing or eliminating the visual presence of the anomaly.

### BRIEF DESCRIPTION OF THE DRAWINGS

FIG. 1 is a perspective view of a portion of the end of a finger and a fingernail.

FIG. 2 is cross section from section lines A-A in FIG. 1.

FIG. 3 is top view showing a chip in a nail coating at the end of a nail.

FIG. 4 is a cross section from lines B-B in FIG. 3.

FIG. 5 is a top view of a nail showing the chip from FIG. 4 repaired according to the present invention.

FIG. 6 is a top view of the nail showing a chip 31 in coating 26.

FIG. 7 is a cross-section along lines C-C taken from FIG. 6.

FIG. 8 is FIG. 7 but with carrier 22 in the chip area 31 with additional indicia show light transmission relative to the carrier medium 22.

### DETAILED DESCRIPTION OF THE PREFERRED EMBODIMENT

The composition of the present invention provides a functional nail filling composition that has incorporated therewith, one or more materials for reflecting color of or equal to an adjacent material. In one embodiment, the composition is effective for filling any anatomical part of the nail or its coating.

The reflective material of the composition is dispersed, dissolved or suspended in a liquid or semi solid carrier. The carrier includes aqueous, organic, or cosolvent mixture of aqueous and organic solvents.

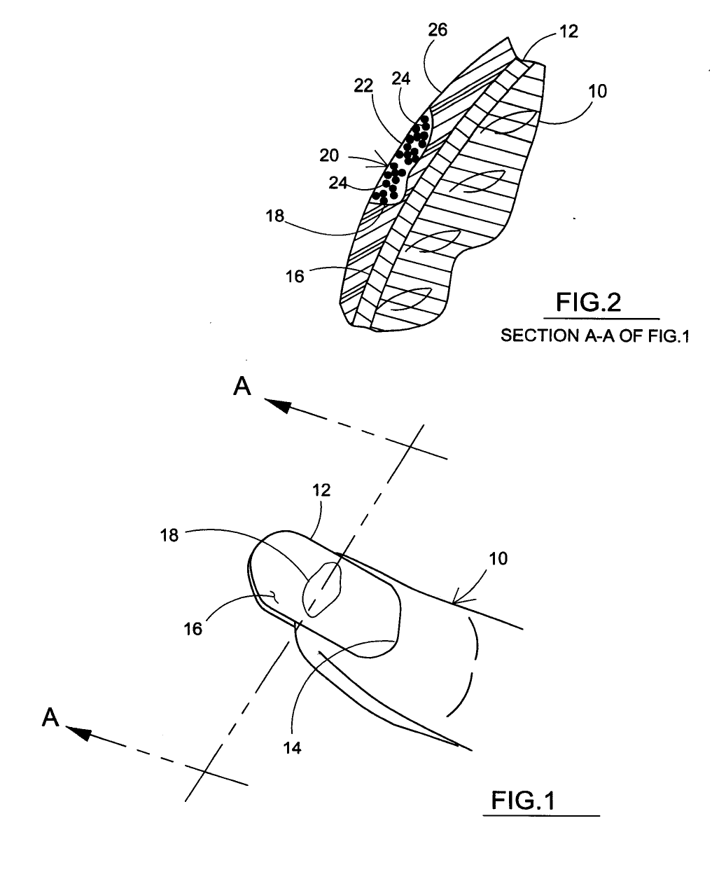

Finger 10 has a fingernail 12 at a distal end. Cuticle 14 is the interface between finger 10 and fingernail 12. Fingernail 12, as shown in FIG. 1, has an upper surface 16 in which a crack 18 has been formed. Crack 18 has cavity 20 defining the boundary of crack 18. Carrier 22 is placed into cavity 20 in one example by using a small brush that transfers the carrier from a container to the cavity. In one embodiment, carrier 22 includes a reflective material 24 incorporated within the carrier such that color from coating layer 26 is reflected outward from the carrier toward the observer. If coating 26 is not present on nail 12, the natural color of upper surface 16 of the nail is reflected by reflective material 24.

Carrier 22 is a composition preferably that evaporates in a approximately 10-120 seconds after application into cavity 20. Carrier 22 also contains an appropriate carrier agent including any of fillers, dispersants, suspending agents, and combinations thereof as desired. In one embodiment, carrier 22 contains a cellulosic compound as a filler. Cellulosic compounds include, but are not limited to, methylcellulose, microcrystalline cellulose, propylcellulose, hydroxymethyl cellulose and hydroxypropyl methylcellulose. In one embodiment, carrier 22 includes keratin compositions similar to naturally occurring fingernail or toenail keratin.

Reflective material 24 is any material, element, compound, or molecule known to reflect color when positioned near a colored surface 16 or 26. Reflective material 24 may include micronized crystalline solids of inorganic salts, known optical coating materials reflecting at least some portion of visible light (i.e. light in the visible spectrum), or materials in which selective reflectivity and transmitivity can be controlled and applied. Reflective material 24 in the present invention is preferably micronized and evenly distributed throughout carrier 22. In one embodiment, reflective material 24 is micronized to 0.1-500 microns. Reflective material 24 need not have a regular or consistent shape, structure, or crystalline form. Any suitable material that reflects surrounding light is usable as reflective material 24.

In another embodiment, carrier 22, comprising a pigment and resin, has a refractive index, n, lower than the refractive index of polish 16. At the carrier polish interface there exists an index where the difference refractive index of the carrier material is lower than the refractive index of the adjoining polish.

In use, the method of the present invention involves identifying at least one nail having an anomaly, such as, but not limited to, a crack;

providing a composition having at least one reflective material and a carrier;

applying said composition to said anomaly;

waiting a set period of time for said composition to cure.

In a preferred embodiment, the composition cures in 10-120 seconds.

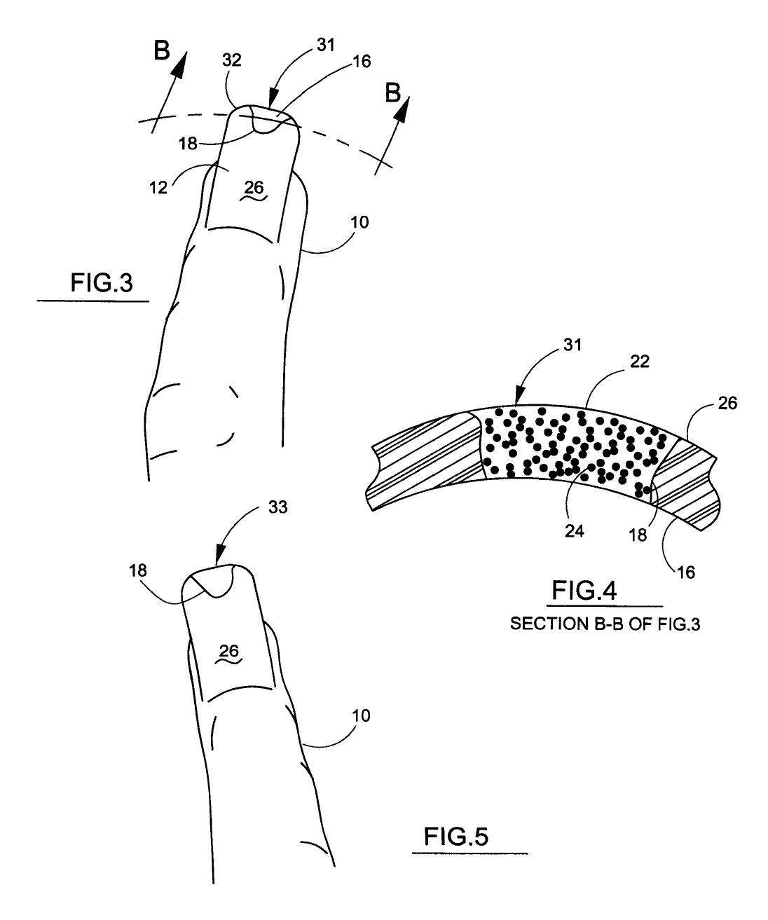

As seen in FIG. 3 the anomaly is a chip 31 at the distal edge 32 of nail 12. As seen in FIGS. 2 and 4, chip 31 is either adjacent coating 26, but generally on top of upper surface 16 of nail 12 or the upper surface of the nail 12 is more exposed. Carrier 22 is applied and placed within chip 31 and the adjoining areas of coating 26 so that the chip 31 is filled. Additionally a polishing cloth which may have a polishing compound can be used to smooth the new surface of the carrier 22 and adjoining polish 26 so the two surfaces are smooth at the point where they meet on the top surface. The new top surface 33 of coating 26 is substantially the same color as the original top surface before the chip as shown on FIG. 5.

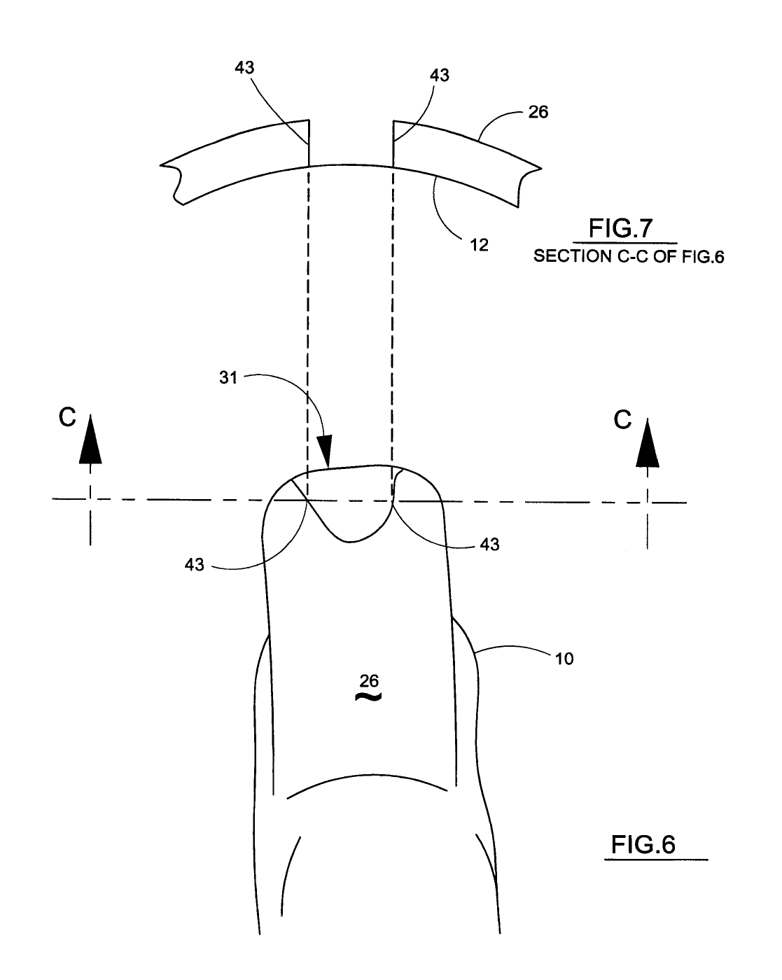

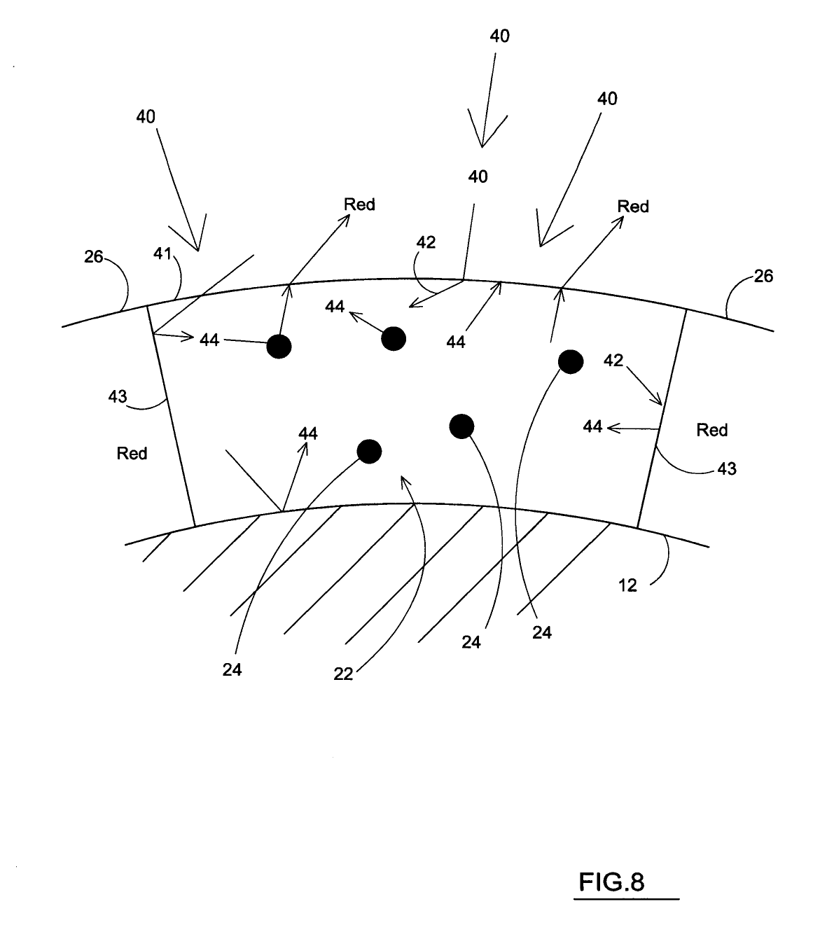

Referring to FIGS. 6, 7 and 8, which shows a cross section of the nail 12, polish or coating 26, and carrier 22, with reflective material 24. Ambient light 40 is shown in a direction-entering carrier 22. At the surface of entry 41, the top of carrier 22, the light bends 42 as is known in the art. Within the carrier, the light reflects from at least 3 surfaces, the internal surfaces 43 between the coating 26 and carrier 22. At this internal surface 43, reflected light 44 reflects off surface 43 back into carrier 22 because of the lower refractive index of the carrier 22 compared to the higher refractive index of polish 26. This reflected light 44 will disperse within carrier 22 and may further reflect from reflective material 24 and from nail surface 12. Eventually reflected light 44 will transmit out of the carrier 22 at surface 41 and become visible red light that an observer will see as identical to or substantially the same color as the red surface 26.

In another alternate embodiment, the carrier 22 will contain certain chemicals that will react upon contact with the previously applied nail polish 16 at initially wall 43. This reaction will cause a color change throughout the carrier 22. Carrier 22 will pick up the color properties and formula from polish 16 and morph into the same color. Therefore, the chip will have the same color as the adjoining polish 12. This reaction will happen whether the chip is located in the center of the nail or other location. The color change of the carrier 22 will occur throughout carrier 22 prior to the carrier 22 setting or drying.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A composition comprising:
a. a carrier;
b. a reflective material, wherein said composition cures to a semi solid or solid after application to a nail anomaly and said reflective material reflects all nearby color giving a visual appearance of said anomaly similar to nearby color.

2. The composition of claim 1 wherein said carrier comprises at least one solvent and at least one carrier agent.

3. The composition of claim 2, wherein said solvent is aqueous, organic, or cosolvent mixtures thereof.

4. The composition of claim 2 wherein said carrier agent is a compound selected from fillers, dispersants, suspending agents, solubilizers, and combinations thereof.

5. The composition of claim 1 wherein said reflective material is any material, element, compound, or molecule known to reflect color of a nearby colored surface when positioned near said colored surface.

6. The composition of claim 1 wherein said reflective material is micronized to 0.1-500 microns.

7. A method of repairing a nail anomaly comprising the steps of
identifying at least one nail having an anomaly, such as, but not limited to, a crack;
providing a composition having at least one reflective material and a carrier;
applying said composition to said anomaly;
waiting a set period of time for said composition to cure.
